(19) United States
(12) Patent Application Publication
(10) Pub. No.: US 2016/0184128 A1
(43) Pub. Date: Jun. 30, 2016

(54) External Mandibular Advancement Splint

(71) Applicant: Louis Kenneth Zweig, Arlington, VA (US)

(72) Inventor: Louis Kenneth Zweig, Arlington, VA (US)

(21) Appl. No.: 14/963,430

(22) Filed: Dec. 9, 2015

Related U.S. Application Data
(60) Provisional application No. 62/124,704, filed on Dec. 31, 2014.

(51) Int. Cl. A61F 5/56

(52) U.S. Cl. CPC A61F 5/56

(57) ABSTRACT

An external mandibular advancement splint is provided for advancing a wearer's jaw forward during sleep, thereby opening the airway and limiting the obstruction causing snoring or sleep apnea. The external mandibular advancement splint includes an external member having one or more arms, an advancing mechanism, and at least one stabilizing strap. The stabilizing strap is attached to a distal end of the one or more arms to anchor the external member on a wearer's mandible. The arms are adapted to engage with at least a portion of a wearer's mandible and each end of the arms are operably connected to the advancing mechanism. The arms and the advancing mechanism create a fulcrum thereby providing tension to the external member. The tension causes the advancing mechanism to pull the mandible in a direction that alleviates airway constriction.

### FIELD

The present subject matter generally relates to an apparatus for preventing snoring and improving breathing during sleep. More particularly, the subject matter relates to an external mandibular advancement splint that has application in the treatment and prevention of snoring and obstructive sleep apnea (OSA).

### BACKGROUND

Snoring and sleep apnea are problems that affect the health and quality of life of millions of people. Approximately 50% of all adults snore at least once per month, and an estimated 10% of all adults have at least mild sleep apnea. Snoring and OSA occur when there is partial or complete occlusion of the upper airway during sleep caused by muscle relaxation and a narrowed pharynx. Sleep apnea, if left untreated, has been associated with hypertension, arrhythmia, congestive heart failure, depression, headaches, diabetes, obesity, and coronary artery disease, as well as decreased quality of life.

In severe cases of sleep apnea, the pharynx closes so much that enough air cannot get through to the lungs. Most often, this is associated with a brief arousal from sleep. The brain quickly reactivates the muscles that hold the throat open, air passes through again, and the brain goes back to sleep. The repetitive episodes of complete or partial blockage of breathing are characteristic of OSA. According to the American Sleep Apnea Association, more than 12 million Americans suffer from sleep apnea and it is estimated that 10 million Americans remain undiagnosed.

Numerous treatments for sleep apnea exist, but all have significant limitations. For example, continuous positive airway pressure (CPAP) devices are considered the gold standard for treatment of OSA, but only about 50% of individuals can tolerate these devices, and the cost can be prohibitive. Intraoral mandibular advancement splints are another common treatment, but these devices can be unsanitary, often expensive, and difficult to use for people with crowns, dentures, irregular mouth or teeth shape, or a strong gag reflex. Surgery is another option, but is invasive, expensive, often ineffective, and is not reversible. Additionally, nasal expiratory positive airway pressure (EPAP) devices utilize the user's own breathing to create positive airway pressure to prevent obstructed breathing. However, nasal EPAP devices require wearers to insert a small valve attached externally to each nostril with hypoallergenic adhesive, and are disposed of nightly, so create unnecessary waste and become very expensive overtime. Thus, it can be seen that there are several problems with the foregoing approaches.

In addition, the use of drugs is undesirable for a number of reasons, including possible dependence on such drugs, as well as adverse side effects related to drug use. Masks with hoses are not well-tolerated and providing a source of pressurized gas can be complex and expensive. Because of all these limitations, there remains a need for non-pharmacological, improved treatment for snoring and OSA.

In view of the foregoing disadvantages inherent in the known types of snoring and OSA treatments, the present subject matter provides an external mandibular advancement splint that provides a non-pharmacological, non-invasive treatment method that prevents snoring and OSA.

According to one embodiment of the present subject matter, the external mandibular advancement splint comprises an external member having one or more arms adapted to engage with at least a portion of a mandible. At least one stabilizing strap is attached to a distal end of the one or more arms to anchor the external member on the mandible. An advancing mechanism is operably connected to the one or more arms of the external member and is configured to pull the mandible in a direction that alleviates airway constriction.

In another exemplary embodiment, the advancing mechanism is a strap that connects the ends of each arm to apply inward pressure on the arms. The strap creates a fulcrum between the arms to pull the mandible forward. The advancing mechanism may either extend around the maxilla or the nasal bone of a user. The advancing mechanism is composed of a firm but flexible material with inherent structural properties to provide appropriate tension to the external member.

According to another embodiment of the present subject matter, the external mandibular advancement splint comprises a U-shaped external member having at least two arms adapted to engage with at least a portion of a mandible. Each arm extends from opposing sides of the central portion of the external member. The central portion includes a hinge member that is pivotally connected to an advancing bar. A spring is disposed between each arm and pulls each arm of the external member inward to alleviate airway constriction.

According to yet another embodiment of the present subject matter, the external member includes an advancing bar located at an apex thereof. The advancing bar pushes a wearer's head posteriorly to alleviate the airway passage. In some embodiments, the advancing bar may rest on the maxilla or nasal bones of a user.

In a further embodiment, the external mandibular advancement splint comprises an external member having one or more arms adapted to support a user's mandible. The distal ends of the one or more arms are attached to a head strap via an adjustable attachment. The adjustable attachment provides a first position and a second position for the external member. In a first position, the upward tension of the head strap allows the gonion of a user to act as a fulcrum, thereby gently pulling the anterior portion of the external member down, opening the wearer's mouth. In a second position, the upward tension of the head strap pulls the anterior portion of the external member upwards, thereby closing the wearer's mouth.

Other objects, features and advantages of the present invention will become apparent from the following detailed description taken in conjunction with the accompanying drawings.

### BRIEF DESCRIPTION OF THE DRAWINGS

Although the characteristic features of the present subject matter will be particularly pointed out in the claims, the subject matter itself and manner in which it may be made and used may be better understood after a review of the following description, taken in connection with the accompanying drawings wherein like numeral annotations are provided throughout.

FIG. 1A shows a front elevation view of the external mandibular advancement splint according to one embodiment.

FIG. 1B shows a side elevation view of the external mandibular advancement splint according to one embodiment.

FIG. 2A shows a front elevation view of the external mandibular advancement splint with a maxillary advancing bar according to one embodiment.

FIG. 2B shows a side elevation view of the external mandibular advancement splint with a maxillary advancing bar according to another embodiment.

FIG. 3A shows a front elevation view of the external mandibular advancement splint with a nasal bone advancing bar according to one embodiment.

FIG. 3B shows a side elevation view of the external mandibular advancement splint with a nasal bone advancing bar according to another embodiment.

FIG. 4A shows a side elevation view of the external mandibular advancement splint in a first position according to one embodiment.

FIG. 4B shows a side elevation view of the external mandibular advancement splint in a second position according to another embodiment.

### DETAILED DESCRIPTION

Reference is made herein to the attached drawings. Like reference numerals are used throughout the drawings to depict like or similar elements of the external mandibular advancement splint. For the purposes of presenting a brief and clear description of the present subject matter, the preferred embodiment will be discussed as used for treating and preventing snoring and obstructive sleep apnea by opening and limiting obstruction of the airway. The figures are intended for representative purposes only and should not be considered to be limiting in any respect.

This present subject matter provides an external mandibular advancement splint designed to advance the wearer's jaw forward during sleep, thereby opening the airway and limiting obstruction causing snoring or obstructive sleep apnea. The external mandibular advancement splint engages with a wearer's mandible and either the maxilla or nasal bone to gently pull the wearer's jaw forwardly and upwardly. In this way, the external mandibular advancement splint prevents a wearer's tongue from blocking an airway and advances the jaw forward to help keep the airway open.

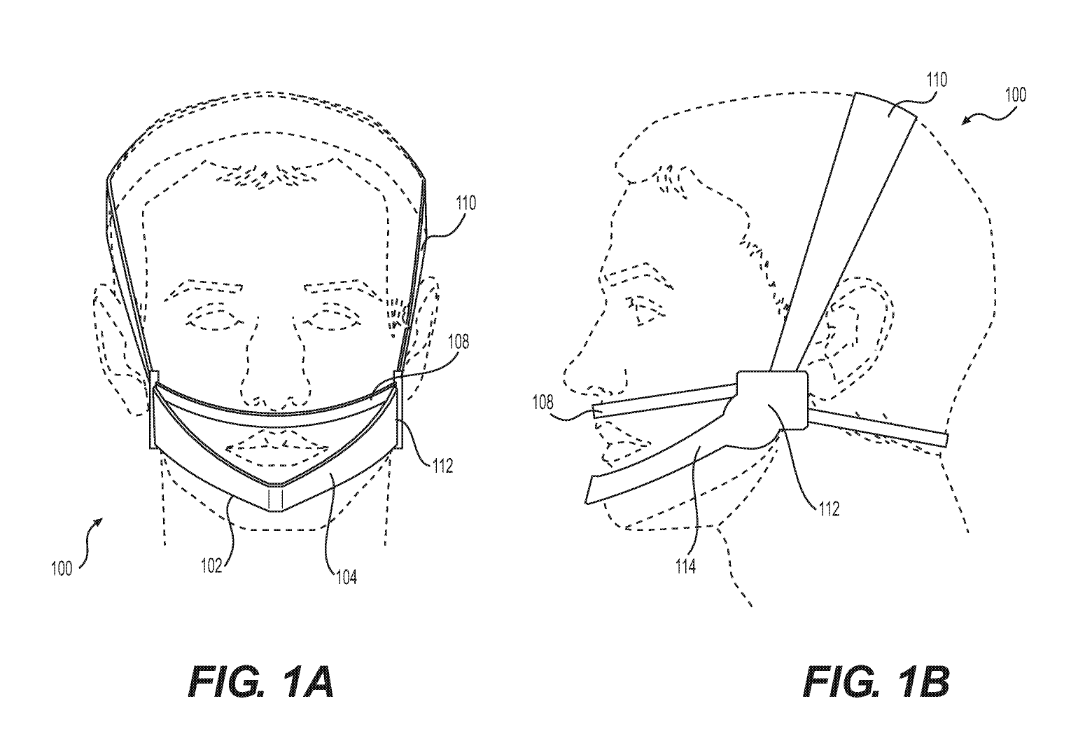

Referring now to FIGS. 1A and 1B, there are shown front and side elevation views of the external mandibular advancement splint, respectively. The external mandibular advancement splint 100 includes an external member 102 having one or more arms 104 adapted to engage with at least a portion of a wearer's mandible. In some embodiments, the arms 104 comprise padded straps that engage the upper jaw between the chin and the lower lip. The distal ends of the arms 104 comprise fingers 112 that are configured to wrap behind the mandible at a gonial angle to provide a secure fit on the mandible.

An advancing mechanism 108 is operably connected to each end of the one or more arms 104. The advancing mechanism 108 is configured to pull the mandible in a direction that alleviates airway constriction. In some embodiments, the advancing mechanism 108 comprises a strap that attaches to each end of the one or more arms 104 and is configured to extend around either a maxilla or a nasal bone of a wearer. The strap of the advancing mechanism 108 is located above the fingers 112, thereby creating a fulcrum between the ends of the arms 104. The advancing mechanism 108 extends across the upper lip of a wearer, thereby connecting the ends of each arm 104. The tension applied by the advancing mechanism 108 gently opens the mouth and advances the jaw forward. More specifically, the force of the advancing mechanism 108 applied to each end of the arm 104 causes gentle pressure against the upper lip while closing the lower jaw and pulling it forward. In another embodiment, as the wearer lies back, the weight of the wearer's head applies pressure to a portion of the device that pushes the external member 102 forward, thereby advancing the jaw.

The external mandibular advancement splint 100 further includes at least one stabilizing strap 110 attached to the distal ends of the one or more arms 104 to anchor the external member 102 on the mandible 106. The device can include one or more stabilizing straps 110 to keep the device anchored in place. In some embodiments, the stabilizing strap 110 extends around the periphery of the skull connecting each end of the arms 104. It is contemplated that stabilizing strap 110 can include a fastener to adjust the length and tension of the external member 102.

According to various embodiments, the external mandibular advancement splint 100 is substantially U-shaped with an advancing mechanism 108 connected to each arm 104 to apply inward pressure and firmly grasp the mandible. The fingers 112 at the end of each arm 104 wrap behind the mandible at the gonion and firmly grasp the mandible bilaterally and posteriorly. The external mandibular advancement splint 100 is then projected forward by applying pressure to either the maxilla or nasal bones of a wearer. As the external mandibular advancement splint 100 extends forward, the mandible is pulled forward as well, thereby advancing the tongue and opening the airway.

The external mandibular advancement splint 100 comprises a firm and flexible plastic that has inherent tension that allows it to bend inwardly, thereby creating enough tension to keep the device in place. In one embodiment, the external mandibular advancement splint 100 is made of a rubber or other suitable flexible plastic material that conforms to the shape of a wearer's head and provides tension.

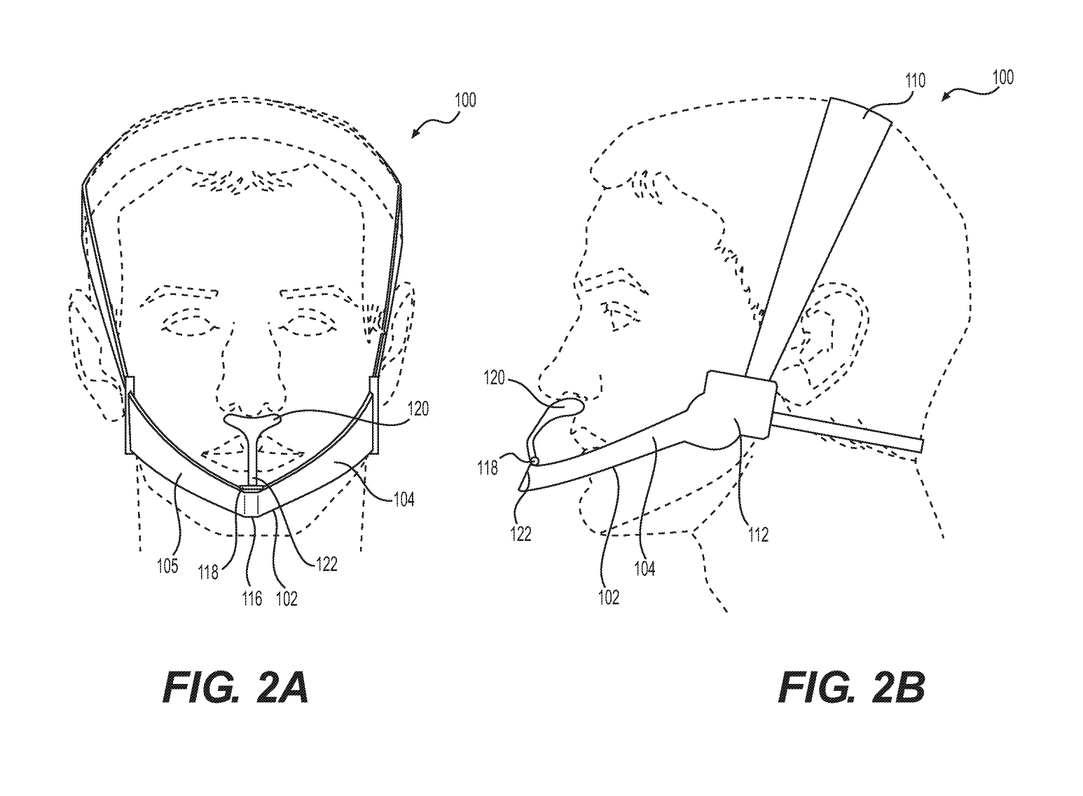

Referring now to FIGS. 2A and 2B, there are shown front and side elevation views of the external mandibular advancement splint with a maxillary advancing bar according various embodiments. The external mandibular advancement splint 100 according to the illustrated embodiment includes a U-shaped external member 102 having at least two arms 104, 105 adapted to engage with at least a portion of a mandible. A hinge member 118 is located at a central portion 116 of the external member 102. Each arm 104, 105 extends from opposing sides of the central portion 116. An advancing bar 120 is pivotally connected to the hinge member 118. The stabilizing strap 110 is attached to distal ends of the arms 104, 105 to anchor the external member 102 on the mandible. A spring 122 is operably connected between the arms to pull each arm 104, 105 of the external member 102 inward to alleviate airway constriction.

The external mandibular advancement splint 100 includes a hinge member 118 at the apex of the central portion 116 with two separate arms 104, 105 extending from opposing sides of the central portion 116. The spring 122 between the two arms 104, 105 pull the arms 104, 105 inward, creating pressure on the jaw and holding the device in place on the wearer's mandible. The spring 122 may also be connected to a safety device, such as a flywheel or similar device, which allows the device to close slowly, so it does not snap shut and injure the wearer. In yet another embodiment of the external mandibular advancement splint 100, the spring 122 connecting the two arms 104, 105 may further include a cylindrical or circular knob that can be tightened by the wearer to adjust the tension provided to each arm 104, 105.

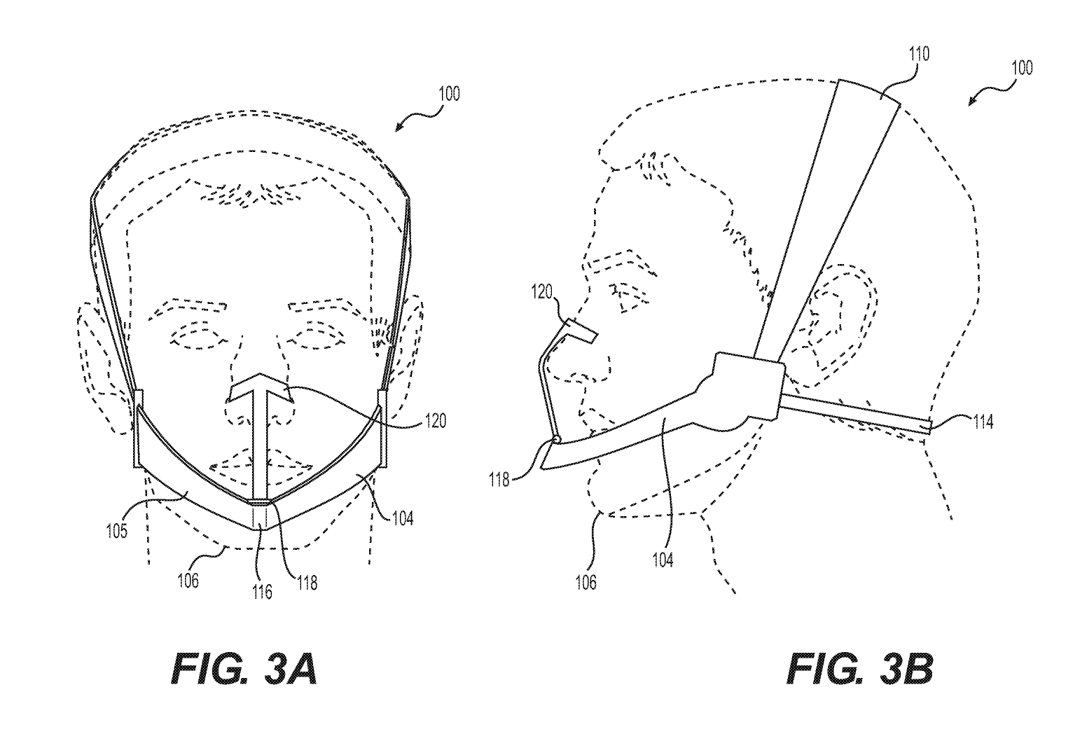

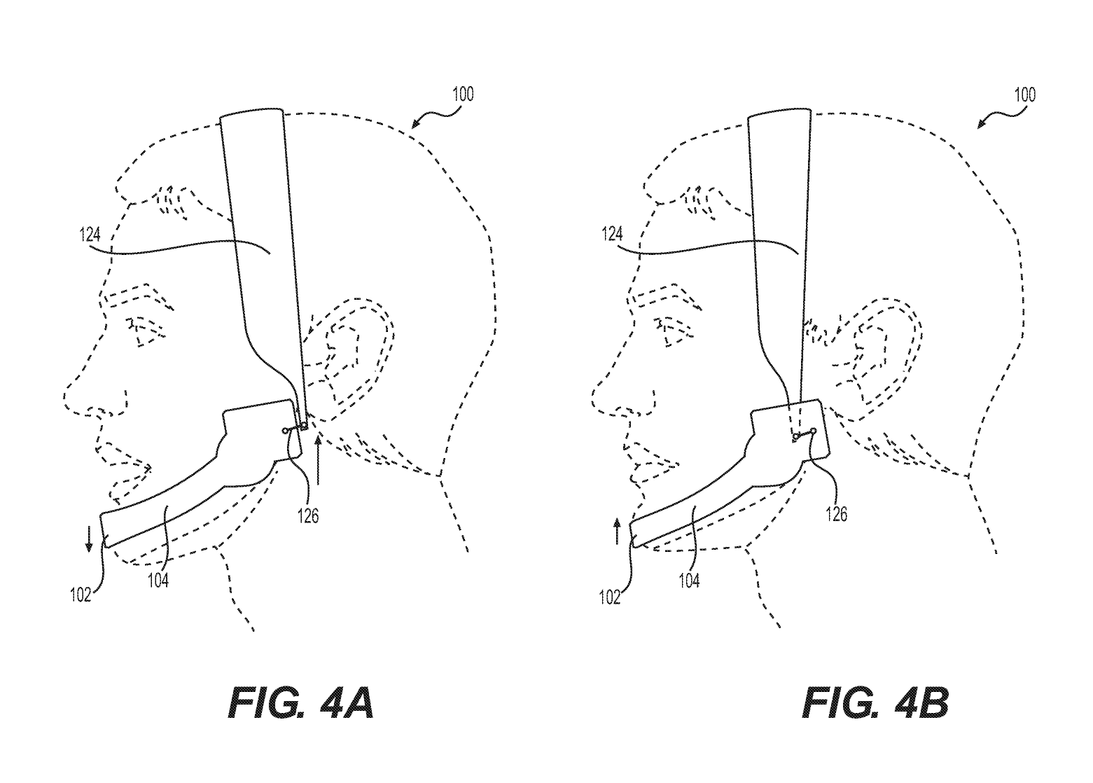

Referring now to FIGS. 3A and 3B, there are shown front and side elevation views of the external mandibular advancement splint with a nasal bone advancing bar according various embodiments. In the illustrated embodiment, the external mandibular advancement splint 100 comprises an advancing bar 120 that contacts a wearer's nasal bone. The advancing bar 120 is configured to apply pressure to the maxilla (FIGS. 3 and 4) or nasal bone of a wearer. The advancing bar 120 is operably connected to the hinge member 118 to allow the advancing bar 120 to move back and forth on the hinge member 118. In one embodiment, the advancing bar 120 is placed at the apex of the central portion 116. The advancing bar 120 may further include a spring in a base portion thereof in order to push the advancing bar 120 posteriorly towards the back of the head. The advancing bar 120 is configured to rest either on the maxilla or nasal bones, depending on the wearer's preference.

The external mandibular advancement splint 100 further includes at least one tension member 114 that keeps the one or more arms 104 in contact with the mandible 106. The tension member 114 connects the one or more arms 104 on either side of the mandible 106 and extends around the skull in any plane to provide inward tension on the one or more arms 104 to keep the external member 102 affixed to the mandible 106. In a preferred embodiment, the tension member 114 is an elongated flexible strap that can adjust the tension force of the arms 104, 105 applied to the mandible of a wearer.

FIGS. 4A and 4B show side elevation views of the external mandibular advancement splint in a first position and a second positon, respectively, according to exemplary embodiments. In the illustrated embodiment, the external mandibular advancement splint 100 comprises an external member 102 having one or more arms 104 adapted to support a user's mandible. The distal ends of the one or more arms 104 are attached to a head strap 124 via an adjustable attachment 126. The head strap 124 anchors the external member 102 on the mandible and provides tension and support to the external mandibular advancement splint 100.

The adjustable attachment 126 operably connects the arms 104 of the external member 102 to the head strap 124. In some embodiments, the adjustable attachment 126 is a hinge member, such as a short bar, to provide a first position and a second position for the external mandibular advancement splint 100, depending on the desired effect. In a first position, the upward tension of the head strap 124 allows the gonion of a user to act as a fulcrum, thereby gently pulling the anterior portion of the external member 102 down, opening the wearer's mouth. In a second position, the upward tension provided by the head strap 124 pulls the anterior portion of the external member 102 upwards, thereby closing the wearer's mouth. In this way, the external mandibular advancement splint 100 provides two configurations, as some people are “mouth-breathers” and some are “nose-breathers”, allowing the wearer to choose the optimal position.

It is contemplated that most or all of the components of the present subject matter can be made of inexpensive materials such as plastic, rubber, or similar materials. The use of such materials is expected to greatly decrease the cost. Further, because the device is adjustable by the patient, it is expected that there will be no need to have custom-fit components.

While certain exemplary embodiments and implementations have been described herein, other embodiments and modifications will be apparent from this description. Accordingly, the various embodiments described are not intended to be limiting, but rather are encompassed by the broader scope of the presented claims and various obvious modifications and equivalent arrangements.

I claim:

1. An external mandibular advancement splint for alleviating airway obstruction, comprising:
an external member having one or more arms adapted to engage with at least a portion of a mandible;
an advancing mechanism operably connected to each end of the one or more arms to create a fulcrum between the ends of the arms;
at least one stabilizing strap attached to a distal end of the one or more arms to anchor the external member on the mandible;
wherein the advancing mechanism is positioned to pull the mandible in a direction that alleviates airway constriction.

2. The external mandibular advancement splint of claim 1, wherein the distal ends of the one or more arms comprise fingers that are configured to wrap behind the mandible at a gonial angle.

3. The external mandibular advancement splint of claim 1, wherein the advancing mechanism is a strap that attaches to each end of the one or more arms and is configured to extend around either a maxilla or a nasal bone.

4. The external mandibular advancement splint of claim 1, further comprising at least one tension member that keeps the one or more arms in contact with the mandible.

5. The external mandibular advancement splint of claim 4, wherein the tension member connects the one or more arms on either side of the mandible and extends around the skull in any plane to provide inward tension on the one or more arms to keep the external member affixed to the mandible.

6. The external mandibular advancement splint of claim 1, wherein the one or more arms have an adjustable length.

7. The external mandibular advancement splint of claim 6, further comprising a mechanism to adjust the tension of the external member by the wearer to keep the one or more arms affixed to the mandible during use.

8. The external mandibular advancement splint of claim 7, wherein the mechanism is a knob or a wheel.

9. The external mandibular advancement splint of claim 1, wherein the advancing mechanism is composed of a firm and flexible material with inherent structural properties to provide tension to the external member.

10. The external mandibular advancement splint of claim 9, wherein the advancing mechanism comprises at least one strap that attaches to the one or more arms and advances the external member by applying pressure to a portion of the body separate from the mandible thereby advancing the jaw.

11. The external mandibular advancement splint of claim 1, wherein the one or more arms apply pressure to another portion of the wearer's body separate from the mandible thereby advancing the jaw.

12. The external mandibular advancement splint of claim 1, wherein the splint utilizes the weight of the wearer's head to apply pressure to the external member, thereby advancing the mandible.

13. The external mandibular advancement splint of claim 1, wherein the splint comprises a mechanism that employs gravity to apply gentle downward pressure to the mental protuberance to open the jaw to allow for better mouth breathing.

14. An external mandibular advancement splint to alleviate airway obstruction, comprising:
a U-shaped external member having at least two arms adapted to engage with at least a portion of a mandible;
a hinge member located at a central portion of the external member, wherein each arm extends from opposing sides of the central portion;
an advancing bar pivotally connected to the hinge member;
at least one stabilizing strap attached to a distal ends of the arms to anchor the external member on the mandible; and
a spring operably connected between the arms, wherein the spring pulls each arm of the external member inward to alleviate airway constriction.

15. The external mandibular advancement splint of claim 14, wherein the advancing bar includes a spring in a base portion thereof configured to push posteriorly.

16. The external mandibular advancement splint of claim 15, wherein the advancing bar rests on the maxilla of a user.

17. The external mandibular advancement splint of claim 15, wherein the advancing bar rests on the nasal bone of a user.

18. An external mandibular advancement splint for alleviating airway obstruction, comprising:
an external member having one or more arms adapted to engage with at least a portion of a mandible;
a head strap attached to a distal end of the one or more arms to anchor the external member on the mandible;
an adjustable attachment coupling the arms of the external member to the head strap, the adjustable attachment arranging the external member in a first position and a second position, whereby, in the first position, the head strap provides upward tension to allow the gonion of a user to act as a fulcrum, thereby gently pulling an anterior portion of the external mandibular advancement splint down opening the wearer's mouth, and whereby, in the second position, the head strap provides upward tension to pull the anterior portion of the external mandibular advancement splint upwards, thereby closing the wearer's mouth.

19. The external mandibular advancement splint of claim 18, wherein the adjustable attachment is a hinge member.

20. The external mandibular advancement splint of claim 19, wherein the hinge member is a short bar.
